(19) United States
(12) Patent Application Publication
(10) Pub. No.: US 2002/0034522 A1
(43) Pub. Date: Mar. 21, 2002

(54) USE OF A STRAIN OF PASTEURELLA HAEMOLYTICA OF A PARTICULAR SEROTYPE FOR PREPARING A VACCINE AGAINST BOVINE PASTEURELLOSIS DUE TO PASTEURELLA HAEMOLYTICA

(75) Inventors: CHRIS ADLAM, PIETREBAIS (BE); DAMIEN SCHREUER, MOFFANS ET VACHERESSE (FR); CATHERINE SCHUHMACHER, LURE (FR)

Correspondence Address: FLYNN THIEL BOUTELL & TANIS PC, 2026 RAMBLING ROAD, KALAMAZOO, MI, 490081699

(73) Assignee: FLYNN, THIEL, BOUTELL, AND TANIS, P.C.

(21) Appl. No.: 09/291,782

(22) Filed: Apr. 14, 1999

(30) Foreign Application Priority Data
Mar. 13, 1998 (CA) 2 232 119

(51) Int. Cl. A61K 39/102

(52) U.S. Cl. 424/255.1

(57) ABSTRACT

A vaccine against bovine pasteurellosis due to Pasteurella haemolytica, characterised in that it comprises a first antigenic substance comprising at least one component chosen from the group consisting of:

(a) The supernatant, concentrated if required, of a bacterial culture of Pasteurella haemolytica serotype A6,

(b) An extract containing capsular antigens of Pasteurella haemolytica serotype A6, and

(d) Whole bacterial bodies of Pasteurella haemolytica serotype A6,

the said supernatant, the said extract and the said bacterial bodies being biologically inactivated.

The invention relates to use of a strain of Pasteurella haemolytica of a particular serotype for preparing a vaccine against bovine pasteurellosis due to Pasteurella haemolytica.

Respiratory diseases of young bovines, mainly of the “infectious enzootic broncho-pneumonia” type, are a major problem in farms producing red and white meat. The resulting mortality, cost of treatment and failure of animal husbandry are the cause of serious financial losses.

These respiratory diseases include bovine pasteurellosis, which is due to two biological germs, Pasteurella haemolytica and Pasteurella mutlocida, the first of these germs being the most frequently encountered in these diseases and the most pathogenic.

To date there are two known biotypes of Pasteurella haemolytica, i.e. biotype A and biotype T. The difference between these two biotypes relates not only to their capacity to ferment sugars (biotype A: arabinose +, trehalose −; biotype T: arabinose −, trehalose +) but also to their characteristics in culture, their resistance to antibiotics, their genome and their pathogenicity.

Also, each biotype A and T can be subdivided into serotypes. To date there are 17 known serotypes (13 for biotype A and 4 for biotype T).

Various authors hitherto have studied the distribution of serotypes (serotyping) of Pasteurella haemolytica, based on samples taken from various species of animals. For example:

H. J. BALL et Coll., Br. Vet. J. (1993), 149,561 showed that in 165 bovine samples from all ages, obtained in North Ireland between 1989 and 1991, the serotype most frequently identified by the method of serotyping by direct haemagglutination was serotype 1 of biotype A or serotype A1 (62 samples) followed in decreasing order by non-typable Pasteurella haemolytica (38 samples), serotype A2 (13 samples), serotype T10 (12 samples), serotype T15 (8 samples), serotype T4 (7 samples), serotypes A8 and A11 (6 samples each), serotypes A6 and A9 (5 samples each) and serotypes A14 and A13 (2 and 1 sample respectively). Note however that in this study, only 33 samples came from the lung, so that there is a doubt regarding the significance of the results of this study. As is known, the other part of the respiratory tract of bovines is not a sterile environment and consequently Pasteurella haemolytica is frequently present in the nasal cavity of healthy animals whereas it is only in sick animals that the germ proliferates and invades the lower part of the respiratory tract;

M. W. ODENTAAL and M. M. HENTON, Journal of Veterinary Research, 62:223-226 (1995) studied the distribution of Pasteurella haemolytica serotypes on the basis of ovine, caprine and bovine samples taken in South Africa over an 8-year period from 1986 to 1994 and showed that in 67 typable samples out of 96 samples, the serotype most frequently identified by the passive haemagglutination method was serotype A1 (38 samples) followed by serotype A14 (8 samples), serotype A6 (6 samples) and serotypes A2 and A15 (5 samples each);

J. L. MARTEL and F. POUMARAT, at the Congress of the Société Francaise de Buiatrie at Paris in 1988, presented a study of the bacteria and mycoplasms associated with respiratory diseases of young bovines in France and showed the prevalence of serotype A1, this species accounting for about two-thirds of Pasteurella samples in sick bovines;

J. L. MARTEL and R. SANCHIS studied the frequency of Pasteurella haemolytica serotypes isolated in bovines in France and presented their results at the Annual Congress of the Société Francaise de Buiatrie on Nov. 29-30, 1995. The strains were serotyped by the passive haemagglutination method. They showed that out of 155 strains of bovine origin studied, 77% were serotype A1 and about 6% were not typable. The other strains, very much in the minority, belong to serotype 2 (8%), 6 (3%), 11 (2%), 14 (1%), 5 and 10 (<1%). However the authors did not specify either the method of sampling the strains or the pathological state of the animal or even the organ from which the samples were taken.

These prior studies therefore clearly show that strains of Pasteurella haemolytica serotype A1 are those most frequently found in bovines and are therefore considered the most virulent. Under these conditions it is not surprising that strains of this serotype have hitherto been most widely used in preparing vaccines against bovine pasteurellosis.

The applicants themselves made an epidemiological study and accordingly serotyped 107 strains of Pasteurella haemolytica of bovine origin from 1992 to 1995. The results of this study are given in the following Table:

| | Serotype | Serotype | Serotype | Serotype | Not | |
|---|---|---|---|---|---|---|
| Sample | A1 | A6 | A2 | A9 | typable | Total |
| ATT | 23 | 16 | 2 | 0 | 2 | 43 |
| | (21.49%) | (14.95%) | (1.86%) | (0%) | (1.86%) | (40.18%) |
| Others £ | 15 | 27 | 2 | 1 | 9 | 54 |
| | (14.01%) | (25.23%) | (1.86%) | (0.93%) | (8.41%) | (50.46%) |
| Unknown $ | 6 | 1 | 1 | 1 | 1 | 10 |
| | (5.60%) | (0.93%) | (0.93%) | (0.93%) | (0.93%) | (9.34% |
| Total | 44 | 44 | 5 | 2 | 12 | 107 |
| | (41.10%) | (41.10%) | (4.67%) | (1.86%) | (11.21%) | |

ATT = Sampling by trans-tracheal suction
£ = Sampling by nasal swabs, nasal pharyngeal swabs or from lung without details
$ = Unknown sampling

These results show that if no distinction is made regarding the method of sampling, i.e. of the place from where the samples were taken, as many strains of serotype A6 as strains of serotype A1 will be found among the strains studied.

Admittedly nasal or nasal-pharyngeal swabs indicate the state of infection of the animal, but it is known that bovines are frequently healthy carriers in the upper respiratory tracts, and an examination of the nasal mucus will not be representative of the lung flora. Note however that there are about 25% samples of serotype A6 against only about 14% samples of serotype A1 from the upper respiratory tract.

The tracheo-bronchial samples are those which really reflect the flora at the pulmonary level and capable of inducing lesions. As shown by line ATT in the Table hereinbefore, serotype A1 accounts for about 21.5% followed by serotype A6, which makes up about 15%. A significant proportion of Pasteurella haemolytica serotype A6 therefore occurs at the pulmonary site, a finding which in completely unexpected manner goes against the conclusions reached by the authors of the previous studies mentioned hereinbefore. This fundamental difference between the findings by the applicants and the results of the prior studies can be explained inter alia by:

The methods of sampling which, in most cases in the study by the applicants, were nasal-pharyngeal swabs or trans-tracheal suction, which have the special feature of enabling samples to be taken right in the lower part of the respiratory tract, whereas in the earlier studies the method of sampling is not specified or mainly relates to sites corresponding to the upper part of the respiratory tract, and

The pathological state of the animals from which samples were taken and which, in the study by the applicants, were all sick animals whereas information on this point was non-existent in some of the prior studies.

In order to confirm the previously-mentioned finding regarding the part played by Pasteurella haemolytica serotype A6 in bovine pasteurellosis, the applicants studied the pathogenicity of Pasteurella haemolytica serotype A6.

Note that hitherto, experimental infections have been successfully produced in pre-ruminants using Pasteurella haemolytica serotype A1.

We therefore made two studies, the aim being to induce pneumonia in young calves by a single injection of Pasteurella haemolytica serotype A6 isolated from the site, in order to prepare an experimental infection model.

More specifically, two three-week calves each received a trans-tracheal injection for introduction into the bronchi of 25 ml of a suspension (in a liquid medium keeping the bacteria alive, e.g. in physiological solution or a culture medium) containing about 10⁸ UFC/ml of a first strain of Pasteurella haemolytica serotype A6 in one case and 10⁹ UFC/ml of the said first strain in the other case. Two other three-week calves were each given a trans-tracheal injection in order to introduce 25 ml of a suspension into the bronchi, the suspension containing about 10⁸ UFC/ml of a second strain of Pasteurella haemolytica serotype A6 in one case and 10⁹ UFC/ml of the said second strain in the second case.

It was confirmed that after about 48 hours the two strains under test from the site induced severe pneumonia, irrespective of the amount of inoculum. This showed the pathogenic nature of Pasteurella haemolytica serotype A6.

Accordingly, contrary to what has been very widely assumed hitherto, Pasteurella haemolytica serotype A1 and also Pasteurella haemolytica serotype A6 are both major pathogenic agents in bovine respiratory tracts, so that of course Pasteurella haemolytica serotype A6 can be used alone or together with Pasteurella haemolytica serotype A1 for preparing a vaccine.

One object of the invention therefore is to use certain specific components of a strain of Pasteurella haemolytica serotype A6, either alone or together with a strain of Pasteurella haemolytica serotype A1, for preparing a vaccine against bovine pasteurellosis due to Pasteurella haemolytica.

The resulting vaccine will be efficient not only against pasteurellosis due to Pasteurella haemolytica serotype A1 but also pasteurellosis due to Pasteurella haemolytica serotype A6. The vaccine will therefore protect the bovine species to a much greater extent than the existing vaccines, which are based on the strain Pasteurella haemolytica serotype A1 only.

Accordingly the invention relates on the one hand to a vaccine against bovine pasteurellosis due to Pasteurella haemolytica, the vaccine being characterised in that it comprises a first antigenic substance comprising at least one component chosen from the group consisting of:

(a) The supernatant, concentrated if required, of a bacterial culture of Pasteurella haemolytica serotype A6,

(b) An extract containing capsular antigens of Pasteurella haemolytica serotype A6, and

(c) Whole bacterial bodies of Pasteurella haemolytica serotype A6,

the said supernatant, the said extract and the said bacterial bodies being biologically inactivated.

The vaccine according to the invention may also comprise a second antigenic substance comprising at least one component chosen from the group made up of:

(a′) The supernatant, concentrated if required, of a bacterial culture of Pasteurella haemolytica serotype A1,

(b′) An extract containing capsular and/or membrane antigens of Pasteurella haemolytica serotype A1,

(c′) Whole bacterial bodies of Pasteurella haemolytica serotype A1, and

(d′) Fractions of the said bacterial bodies,

the said supernatant, the said extract, the said bacterial bodies and the said fractions having been biologically inactivated.

Since the said whole bacterial bodies may have some intrinsic toxicity, according to the invention they are preferably present in small proportions in the vaccine.

In a variant of the invention, the said first antigenic substance is the biologically inactivated, concentrated supernatant of a culture of Pasteurella haemolytica serotype A6.

In another variant of the invention, the said second antigenic substance is the biologically inactivated, concentrated supernatant of a culture of Pasteurella haemolytica serotype A1.

In a particularly preferred variant of the invention, the vaccine comprises a first antigenic substance consisting of the biologically inactivated, concentrated supernatant of a culture of Pasteurella haemolytica serotype A6 and a second antigenic substance consisting of the biologically inactivated, concentrated supernatant of a culture of Pasteurella haemolytica serotype A1.

Depending on the desired efficacy, the vaccine may also comprise an added leucotoxin of Pasteurella haemolytica other than the leucotoxin possibly already present in the said first antigenic substance and/or in the said second antigenic substance. The added leucotoxin can e.g. be recombinant leucotoxin or leucotoxin obtained by purification from a supernatant of a bacterial culture of Pasteurella haemolytica serotype A6 and/or Pasteurella haemolytica serotype A1.

In yet another variant, the extract containing capsular antigens of Pasteurella haemolytica serotype A6 and the extract containing capsular antigens of Pasteurella haemolytica serotype A1 are respectively obtained by extraction from cells of Pasteurella haemolytica serotype A6 or A1, using an aqueous solution of a mineral or organic salt.

Note that the said capsular antigens are of polysaccharide type and the salt used for extraction is e.g. sodium chloride or sodium salicylate.

Inactivation of the said supernatant(s), extract(s) and whole bacterial bodies and of the said fractions of the said bacterial bodies can be effected by any means well-known in the prior art, e.g. chemical inactivation, preferably by formaldehyde or phenol, or thermal inactivation.

In order to increase the efficacy of the vaccine, it is advantageous, though not necessary, to incorporate at least one immunisation adjuvant in the vaccine according to the invention. The adjuvant can be any immunisation adjuvant conventionally used in the prior art, one example being aluminium hydroxide in gel form (e.g. the substance available under the trade mark ALHYDROGEL® from the Danish company SUPERFOS A/S), or a saponin such as quillaia saponin (e.g. that available under the trade mark QUIL-A from the Danish company SUPERFOS A/S). The adjuvant can be used at various concentrations which can easily be found by the skilled man.

In the case where the vaccine comprises a concentrated supernatant of a culture of Pasteurella haemolytica serotype A6, a concentrated supernatant of a culture of the strain Pasteurella haemolytica serotype A1 and if required an added leucotoxin of Pasteurella haemolytica, the vaccine has been found highly efficacious when the quantity of the first concentrate is such that its 50% leucotoxic activity is at least eight units and the quantity of the second concentrate is such that its 50% leucotoxic activity is at least eight units.

The definition of 50% leucotoxic activity will be explained hereinafter, it being specified that the threshold activities of eight units hereinbefore are given only by way of illustration of the invention, and activities below these thresholds are fully covered by the present invention.

The invention also extends to any concentrated supernatant of a bacterial culture of Pasteurella haemolytica serotype A6, whether or not the concentrate or supernatant is biologically inactivated.

The invention also extends to a method of preparing a vaccine in which the first and the second antigenic substances are concentrated supernatants of bacterial culture. The process preferably comprises the following operations:

(a) Cultivating a mother-strain of Pasteurella haemolytica serotype A6,

(b) Separate cultivation of a mother strain of Pasteurella haemolytica serotype A1,

(c) Separation by filtration of at least a part of the bacterial cells of the supernatant obtained in operation (a) hereinbefore,

(d) Separation by filtration of at least a part of the bacterial cells of the supernatant obtained by operation (b) hereinbefore,

(e) Concentration of the supernatant obtained by operation (c) and of the supernatant obtained by operation (d) hereinbefore,

(f) Mixing the concentrates obtained by operation (e) hereinbefore in suitable proportions,

(g) Biological inactivation of the two concentrates obtained in operation (e) hereinbefore, before or after mixing them in operation (f) hereinbefore,

(h) Addition if required of one or more immunisation adjuvants to the mixture of inactivated concentrates obtained previously,

(i) Addition if required of Pasteurella haemolytica leucotoxin and

(j) Adjustment if required of the pH of the mixture obtained in operation (i) hereinbefore to the desired value.

The invention also extends to a method of preparing a vaccine wherein the first and the second antigenic substances are extracts containing capsular antigens. One such method preferably comprises the following operations:

(a) Cultivating a mother strain of Pasteurella haemolytica serotype A6 and separate cultivation of a mother strain of Pasteurella haemolytica serotype A1,

(b) Collecting the bacterial cells obtained in operation (a) hereinbefore,

(c) Extraction of the cells collected in operation (b) respectively, using an aqueous solution of a mineral or organic salt,

(d) Elimination of the cellular material in order to recover an aqueous extract containing capsular antigens of Pasteurella haemolytica serotype A6 and an aqueous extract containing capsular antigens of Pasteurella haemolytica serotype A1,

(e) Purification of each extract,

(f) Mixing the purified extracts,

(g) Biological inactivation of the said extracts before or after mixing them in operation (f) hereinbefore,

(h) Addition if required of one or more immunisation adjuvants to the mixture of inactivated extracts obtained previously,

(i) Addition if required of Pasteurella haemolytica leucotoxin and

(j) Adjustment, if required, of the pH of the mixture obtained in operation (i) hereinbefore to the desired value.

Advantageously in the said last process:

The mother strains are cultivated as described hereinafter,

The bacterial cells are collected by centrifuging or filtration,

Extraction is by means of an aqueous solution of sodium chloride or sodium salicylate (preferably at a concentration of about 2.5%) with agitation (at 22° C. or more),

Cellular material is eliminated by high-speed centrifuging and the aqueous extracts are recovered by dialysis against distilled water, and

The said extracts are purified by enzymatic treatment (ribonuclease, deoxyribonuclease and/or proteinase K) in order to eliminate traces of nucleic acids and undesirable proteins, followed by precipitation of the capsular antigens (polysaccharides) by using e.g. three volumes of 95% ethanol (or any other suitable solvent such as acetone), re-dissolving the precipitate formed in water, then re-precipitation in cetyl trimethyl ammonium bromide, after which the precipitated complex is dissolved in 2 M NaCl and subjected to thorough dialysis against water, ultracentrifuging (e.g. at 105 000× g) to eliminate undesirable liposaccharides, and final purification by gel filtration or ion exchange.

Note also that additional recovery of capsular antigens can be obtained as follows: the cellular material eliminated during operation (d) mentioned hereinbefore is subjected to extraction by a hot mixture of phenol and water. The resulting aqueous phase is then dialysed against water until free from phenol, then freeze-dried. The freeze-dried product is purified as hereinbefore, i.e. by ultracentrifuging and gel filtration or ion exchange.

The invention will now be illustrated by the following technical details given without in any way limiting the invention. These details relate to production and study of the efficacy of a vaccine in which the first and second antigenic substances are supernatants of bacterial cultures of Pasteurella haemolytica serotype A6 and Pasteurella haemolytica serotype A1.

I/ Production of a Vaccine

1/ Strains Used

Use was made of a strain of Pasteurella haemolytica serotype A1 and of a strain of Pasteurella haemolytica serotype A6, both of bovine origin, the serotype being obtained inter alia by the well-known method of passive haemagglutination.

Note however that use can be made of any strain of Pasteurella haemolytica serotype A6 and any strain of Pasteurella haemolytica serotype A1, the only condition being that they should be serotype A6 and serotype A1 respectively. Thus, use can be made of any strain from the site after checking, by a conventional serotyping operation, that they belong to the desired serotype or from a collection [such as the American Type Culture Collection (ATCC)].

2/ Multiplication of Strains

Each strain is used to seed a gelose (DSA=Dextrose Starch Agar) in Petri dishes, with incubation at 37° C. under CO₂ (5%) until colonies visible to the naked eye appear (generally in 12 to 14 hours). Each resultant gelose is used to seed a gelose medium (DSA) poured into cell culture bottles, after which the gelose medium is incubated for 15 to 18 hours at 37° C. in a 5% CO₂ atmosphere.

3/ Production Culture

The suspension harvested for each strain from the bottles of cell culture is then used to seed a fermenter in which the culture medium is medium RPMI 1640 containing 5% of DSB (Dextrose Starch Broth) or a heart-brain infusion broth or a DSB broth or a solid culture medium well-known in the art in question. Cultivation at 37° C. is continued until the end of the logarithmic growth phase. Other culture mediums can be used from among those at present available in the market.

These operations can be repeated until the desired quantity of supernatant is obtained.

4/ Harvesting

For each strain, the bacterial cells are then separated from the supernatant e.g. by filtration on membranes having a mesh opening of 0.22 μm.

5/ Determination of Leucotoxin

Since the efficacy of the vaccine depends inter alia on its content of leucotoxins, which are antigens produced during cultivation of the Pasteurella haemolytica strains used, it is necessary to subject the said supernatants to concentrations sufficient to obtain the desired leucotoxic activity. The activity is therefore determined in the supernatants in order to find the concentrations to which they must be subjected to obtain the desired final leucotoxic activity.

The method of determination is as follows:

The leucotoxic activity is determined by a test on microplate using cells BL3 (for Bovine Leukemia Cell from the ATCC collection under code 8037-CRL). Equivalent cells sensitive to leucotoxin may also be used.

The cells are incubated (1 hour at 37° C.) in the presence of various dilutions of the sample (supernatant) for determination (pure, ½, ¼, ⅛, 1/16 . . . 1/64). The cells surviving at the end of the incubation period are detected by staining with neutral red. After solubilisation of the cells, the colour is titrated in a spectrophotometer at 550 nm. The percentage of toxic activity is determined for each dilution as follows:

$$\% toxicity = \frac{A - B}{A} \times 100$$

in which

A=average optical density of 4 control wells containing the culture medium only (since this preparation is not toxic, all the BL3 cells survive), and

B=average optical density of 4 test wells (sample under test).

The percentage toxicity is calculated for each dilution of the sample for determination.

It is found that the toxicity decreases in proportion as the sample is diluted (since the leucotoxin responsible for toxicity is diluted).

If the toxicity is shown graphically in dependence on the dilution, the resulting curve can be used to deduce the strongest dilution of the sample which still gives at least 50% toxicity.

If for example the last dilution still giving at least 50% toxicity is the ½ dilution, the activity of the tested sample (the supernatant before concentration) will be 2 units (i.e. the reciprocal of the dilution). This means that if a value of 8 units is desired for the leucotoxic activity of the vaccine, the supernatant will have to be concentrated between 4 and 8 times in order to obtain the desired strength of 8 units.

6/ Concentration of the Supernatant

The supernatant mentioned in Sections 4/ and 5/ hereinbefore is concentrated to the desired extent by conveying it over membranes having a porosity corresponding to molecular weights of 1 to 10 kD.

7/ Biological Inactivation

Each resulting concentrate (corresponding to the strain of serotype A1 and corresponding to the strain of serotype A6) is then inactivated by adding a 40% aqueous solution of formaldehyde and incubating at 370° C. with agitation for at least 24 hours. The amount of aqueous formaldehyde solution will usually be from 0.1 to 0.5% (V/V) relative to the concentrate. The formaldehyde is then neutralised by adding a solution of sodium metabisulphite.

8/ Mixing

The resulting two inactivated concentrates are then mixed in the desired proportion. Next, ALHYDROGEL is added (e.g. in the proportion of 7.5 mg/ml corresponding to 25 volumes of 3% aqueous gel of aluminium hydroxide per 75 volumes of vaccine without gel) and QUIL-A (e.g. in the proportion of 0.05 mg/ml corresponding to 0.0033 ml of a mother solution of QUIL-A at 15 mg/ml water per ml of final vaccine) and the pH is adjusted to 6.5-8.0 with a 7.5% aqueous solution of sodium bicarbonate, soda or hydrochloric acid followed by packaging in sterile ampoules.

9/ An Example of the Vaccine Composition

One example of a vaccine composition according to the invention is as follows:

36% by volume of concentrate, inactivated and neutralised, obtained from a culture of Pasteurella haemolytica serotype A1 which before inactivation had a 50% leucotoxic activity of at least 8 units,

36% by volume of concentrate, inactivated and neutralised, obtained from a culture of Pasteurella haemolytica serotype A6 which before inactivation had a 50% leucotoxic activity of at least 8 units,

25% by volume of 3% aqueous gel of aluminium hydroxide,

0.33% by volume of mother solution of QUIL-A and

2.67% by volume of a 7.5% solution of sodium bicarbonate.

II/ Study of the Efficacy of the Vaccine

A/ Demonstration that a vaccine containing supernatants of culture of a strain of Pasteurella haemolytica serotype A6 and a strain of Pasteurella haemolytica serotype A1 respectively is capable of protecting pre-ruminant calves against a test with virulent Pasteurella haemolytica serotype A6 organisms.

In this demonstration, groups of young calves were vaccinated intramuscularly with two doses of vaccine preparation or placebo as follows:

Group 1: Placebo

Group 2: Commercial vaccine

Group 3: A vaccine according to the invention (a 75% diluted mixture of a concentrate of supernatant from a bacterial culture of the strain Pasteurella haemolytica serotype A6 and a concentrated supernatant from a bacterial culture of the strain Pasteurella haemolytica serotype A1),

Group 4: The vaccine according to the invention, i.e. a mixture of a concentrated supernatant of a bacterial culture of the strain Pasteurella haemolytica serotype A6 and a concentrated supernatant of a bacterial culture of the strain Pasteurella haemolytica serotype A1.

The vaccinations were made with intervals of 21 days between injections and all the animals received intra-tracheally a culture of Pasteurella haemolytica serotype A6, 7 days before the second injection of the vaccine (or the placebo).

The dose was 25 ml of the said culture at the logarithmic stage and contained 1.7×10⁸ cells per ml. Before and after receiving the culture, the animals were clinically evaluated in accordance with an evaluation system and after deaths, if any, the lungs were examined in order to estimate the extent of infection.

The results are given in Table 1 hereinafter:

TABLE 1

| | | Clinical | | | | |
|---|---|---|---|---|---|---|
| | | evaluation | Evaluation of lungs | | Infected | Weight of |
| Group | Deaths | (*) | Gravity/Surface area | | lobes | lungs (kg) |
| 1 | 3/10 | 40 | 21.5 | 13.8 | 5.8 | 1.36 |
| 2 | 2/8 | 29 | 12.1 | 12.3 | 5.0 | 1.32 |
| 3 | 1/9 | 21 | 13.2 | 10.1 | 4.3 | 1.17 |
| 4 | 1/10 | 19 | 11.2 | 8.2 | 4.6 | 0.94 |

(*) The results are averages for the entire group

This Table shows that in terms of mortality, clinical evaluation, evaluation of the lungs and weight of the lungs, the vaccines according to the invention (groups 3 and 4) are better than the placebo (group 1). The commercial vaccine (group 2) was also better than the placebo but was not as efficacious as the vaccines according to the invention. Note that the commercial vaccine did not contain the serotype A6 component.

B/ Demonstration that a vaccine containing the culture supernatants of a strain of Pasteurella haemolytica serotype A6 and a strain of Pasteurella haemolytica serotype A1 respectively is capable of protecting ruminant calves against a test with a virulent Pasteurella haemolytica serotype A6 organism.

A study on a smaller scale was made in order to test the protective capacity of the vaccine according to the invention on groups of older calves. It is often difficult to reproduce pasteurellosis in these animals, since there is natural exposure to Pasteurella haemolytica organisms, which results in some resistance to the test. However the test did produce disease, which again demonstrated the virulence of the serotype A6 strain used for the test.

Two groups of 4 ruminant calves were given two intramuscular doses either of vaccine according to the invention (group 1) or of placebo (group 2). Doses of vaccine or placebo were administered at intervals of 3 weeks and, 2 weeks after administration of the second dose of vaccine or placebo, the animals were intra-tracheally tested with 40 ml of a culture at the logarithmic stage of a Pasteurella haemolytica serotype A6 strain and containing 1.35×10⁹ organisms per ml. The animals were killed one week after the test. They were clinically followed before and after the test, and the lesions in their lungs were described and evaluated by autopsy.

Two additional calves (group 3) served as non-vaccinated, non-tested controls. The results are given in Table 2 hereinafter.

TABLE 2

| | | | | | Total |
|---|---|---|---|---|---|
| | Evaluation of lungs | | Infected | Weight of | clinical |
| Group | Gravity-surface area | | lobes | lungs (g) | evaluation |
| 1 | 9.8 ± 4.5 | 7.0 ± 2.9 | 3.0 ± 1.4 | 2521 ± | 4 |
| | | | | 566.8 | |
| 2 | 22.8 ± 10.0 | 16.0 ± 6.5 | 6.0 ± 2.2 | 3389 ± | 6 |
| | | | | 1249.0 | |
| 3 | 5.0 ± 7.0 | 2.5 ± 3.5 | 2.5 ± 3.5 | 2083 ± | 0 |
| | | | | 97.6 | |

Note that in this experiment, all animals survived the test.

As shown by the extent of damage to the lungs and the clinical responses, the animals in group 1 which received the vaccine according to the invention were protected by comparison with the animals in group 2 which received the placebo.

C/ Conclusion

The study hereinbefore shows that serotype A6 of Pasteurella haemolytica is virulent, in the case both of pre-ruminant calves and ruminant calves.

A vaccine containing the supernatant, concentrated if required, of a culture of Pasteurella haemolytica serotypes A6 and A1 protected pre-ruminant and ruminant calves after being tested with Pasteurella haemolytica serotype A6.

1. A vaccine against bovine pasteurellosis due to Pasteurella haemolytica, characterised in that it comprises a first antigenic substance comprising at least one component chosen from the group consisting of:
(a) The supernatant, concentrated if required, of a bacterial culture of Pasteurella haemolytica serotype A6,
(b) An extract containing capsular antigens of Pasteurella haemolytica serotype A6, and
(c) Whole bacterial bodies of Pasteurella haemolytica serotype A6,
the said supernatant, the said extract and the said bacterial bodies being biologically inactivated.

2. A vaccine according to claim 1, characterised in that it also comprises a second antigenic substance comprising at least one component chosen from the group made up of:
(a′) The supernatant, concentrated if required, of a bacterial culture of Pasteurella haemolytica serotype A1,
(b′) An extract containing capsular and/or membrane antigens of Pasteurella haemolytica serotype A1,
(c′) Whole bacterial bodies of Pasteurella haemolytica serotype A1, and
(d′) Fractions of the said bacterial bodies,
the said supernatant, the said extract, the said bacterial bodies and the said fractions being biologically inactivated.

3. A vaccine according to claim 1 or 2, characterised in that the said first antigenic substance comprises the supernatant of a culture of Pasteurella haemolytica serotype A6, the supernatant being concentrated and biologically inactivated.

4. A vaccine according to claim 2, characterised in that the said second antigenic substance comprises the supernatant of a culture of Pasteurella haemolytica serotype A1, the supernatant being concentrated and biologically inactivated.

5. A vaccine according to claim 1 or 2, characterised in that it also comprises an added leucotoxin of Pasteurella haemolytica other than the leucotoxin, if any, already present in the said first antigenic substance and/or the said second antigenic substance.

6. A vaccine according to claims 1 or 2, characterised in that the extract containing capsular antigens of Pasteurella haemolytica serotype A6 and the extract containing capsular antigens of Pasteurella haemolytica serotype A1 are respectively those obtained by extraction of cells of Pasteurella haemolytica serotype A6 or Pasteurella haemolytica serotype A1, using an aqueous solution of a mineral or organic salt.

7. A vaccine according to claim 1 or 2, characterised in that the said supernatant(s), extract(s) and whole bacterial bodies and the said fractions of bacterial bodies have been inactivated by formaldehyde or phenol.

8. A vaccine according to claim 1 or 2, characterised in that it also comprises at least one immunisation adjuvant.

9. A vaccine according to claim 8, characterised in that the said immunisation adjuvant is chosen from the group consisting of a saponin and aluminium hydroxide in gel form.

10. A concentrate of the supernatant of a bacterial culture of Pasteurella haemolytica serotype A6, the concentrate or the supernatant being inactivated or not.

11. A method of preparing the vaccine according to any of claims 1 to 9 wherein the first and the second antigenic substances are concentrated supernatants of bacterial culture, characterised in that it comprises the following operations:
(a) Cultivating a mother strain of Pasteurella haemolytica serotype A6,
(b) Separate cultivation of a mother strain of Pasteurella haemolytica serotype A1,
(c) Separation by filtration of at least a part of the bacterial cells of the supernatant obtained in operation (a) hereinbefore,
(d) Separation by filtration of at least a part of the bacterial cells of the supernatant obtained by operation (b) hereinbefore,
(e) Concentration of the supernatant obtained by operation (c) and of the supernatant obtained by operation (d) hereinbefore,
(f) Mixing the concentrates obtained by operation (e) hereinbefore in suitable proportions,
(g) Biological inactivation of the two concentrates obtained in operation (e) hereinbefore, before or after mixing them in operation (f) hereinbefore,
(h) Addition if required of one or more immunisation adjuvants to the mixture of inactivated concentrates obtained previously,
(i) Addition if required of Pasteurella haemolytica leucotoxin and
(j) Adjustment if required of the pH of the mixture obtained in operation (i) hereinbefore to the desired value.

12. A method of preparing the vaccine according to any of claims 1 to 9, wherein the first and the second antigenic substances are extracts containing capsular antigens, characterised in that it comprises the following operations:
(a) Cultivating a mother strain of Pasteurella haemolytica serotype A6 and separate cultivation of a mother strain of Pasteurella haemolytica serotype A1,
(b) Collecting the bacterial cells obtained in operation (a) hereinbefore,
(c) Extraction of the respective cells collected in operation (b), using an aqueous solution of a mineral or organic salt,
(d) Elimination of cellular material in order to recover an aqueous extract containing capsular antigens of Pasteurella haemolytica serotype A6 and an aqueous extract containing capsular antigens of Pasteurella haemolytica serotype A1,
(e) Purification of each extract,
(f) Mixing the purified extracts,
(g) Biological inactivation of the said extracts before or after mixing them in operation (f) hereinbefore,
(h) Addition if required of one or more immunisation adjuvants to the mixture of inactivated extracts obtained previously,
(i) Addition if required of Pasteurella haemolytica leucotoxin and
(j) Adjustment, if required, of the pH of the mixture obtained in operation (i) hereinbefore to the desired value.
